(19) United States
(12) Patent Application Publication
(10) Pub. No.: US 2010/0274298 A1
(43) Pub. Date: Oct. 28, 2010

(54) CASPER PIN APPARATUS AND METHOD OF USE

(76) Inventor: David C.M. Schiff, Vallejo, CA (US)

Correspondence Address: DERGOSITS & NOAH LLP, Three Embarcadero Center, Suite 410, SAN FRANCISCO, CA, 94111, US

(21) Appl. No.: 12/428,239

(22) Filed: Apr. 22, 2009

(51) Int. Cl. A61B 17/86; A61F 5/00

(52) U.S. Cl. 606/329; 606/86 A

(57) ABSTRACT

Casper pins have a single uniform length and are inserted to different depths into vertebrae using different depth insertion tools. The Casper pins have a sharp threaded tip and a drive head at the opposite end. The Casper pins are used with insertion tools that are each marked with a different insertion depth. The insertion tools each have a tubular front section and a driver within the tubular section and a handle for rotating the insertion tool. Once the proper insertion depth is determined, the Casper pin is placed into the corresponding insertion tool. Using the insertion tool, the tip of the Casper pin bores into the vertebra and when the designated insertion depth is reached, the driver of the insertion tool is separated from the driver head of the Casper pin. Thus, the Casper pin cannot be inserted beyond the designated insertion depth of the insertion tool. The process is repeated for the adjacent vertebrae so that two Casper pins extend from the patient. A distractor is used to separate the vertebrae so the surgeon can operate on the disc. Once the disc operation is completed, the distractor is removed and a plate is placed over the Casper pins and moved against the vertebrae. The Casper pins are replaced with screws to hold the plate in place against the vertebrae. With the plate in secured in place, the incision cut into the patient can be closed so the spine is allowed to heal.

### BACKGROUND

Casper pins are typically used during neck operations such as discectomies. During the surgery, the patient's skin is incised and the spine is exposed. Before the Casper pins are inserted into the vertebra, a needle is inserted into the disc for localization of the precise vertebral level. X-rays are taken and the vertebral anteroposterior (a-p) depth, is known from the magnetic resonance imaging machine (MRI) measuring tool, or estimated to determine the proper Casper pin length for the patient. Casper pins having the selected insertion depths are placed in adjacent vertebrae on opposite sides of the damaged disc.

With reference to FIG. 1, a Casper pin 101 has a tip 103, screw threads 105, a hexagonal nut 107 welded onto or formed on the pin 101 and an extension portion 109. The insertion depth D is the distance between the tip of the Casper pin and the front shoulder of the nut 107. The Casper pins come in different insertion depths including 14 mm and 16 mm. Based upon the above methods, the Casper pin having the proper insertion depth D is selected. A special wrench is used to insert the Casper pin 101 into the vertebra. With reference to FIGS. 1 and 2, the hollow wrench 201 has a tubular shaft 205, an inner hexagonal surface 203 and a handle 209. The Casper pin 101 is inserted into the tubular shaft 205 of the wrench 201 until the nut 107 engages the internal hexagonal surface 203 at the front edge of the shaft 205. The tip 103 of the Casper pin 101 is then placed on the vertebra and the wrench 201 is rotated in a clockwise direction which causes the threads 105 to bore into the bone. The tip 103 of the Casper pin 101 is screwed into the vertebra until the nut 107 contacts the vertebra and prevents the Casper pin 101 from being inserted any deeper. The described process is repeated to insert a second Casper pin 101 in the adjacent vertebra.

Once the two Casper pins 101 are inserted into the adjacent vertebrae, a distractor is used to separate the two vertebrae so the surgeon can access the damaged disc. With reference to FIGS. 1 and 3, the distractor 301 has two tubes 305 that are placed over the back ends of the two Casper pins 101. The distractor 301 can also include a rack and pinion gear mechanism 307 that expands to separate the tubes 305 and pull the adjacent vertebrae apart. The distractor 301 also includes a locking mechanism 309 that prevents the rack and pinion gear mechanism 307 from contracting. With the distractor 301 locked in the expanded position to keep the vertebrae separated, the surgeon can perform the necessary procedures on the damaged disc. During a discectomy, the damaged disc is removed and a cage, a bone graft or any other structure that the surgeon chooses, is placed between the vertebrae.

After the surgical procedures are completed, the locking mechanism 309 is released and the vertebrae contract the opposite sides of the cage, bone graft or other structure as a result of muscle tension, and the structure placed into the disc space is compressed. The Casper pins 101 are unscrewed so that they can be removed. The removal of the pins 101 will result in bleeding from the holes formed in vertebrae. The surgeon will estimate the distance between the holes formed in the vertebrae and select an appropriate length plate and screws by trial and error and sometimes the use of X-ray to view the plate length and position on the operated vertebrae. The screws are tightened to secure the plate to the vertebrae.

There are several drawbacks to the described procedure. When the Casper pins are removed, there can be a significant amount of blood from the holes in the vertebra in addition to the bleeding as a result of the disc removal. This bleeding often obscures the implant site and it can be difficult to properly size the plate length. This diminished visibility can make it difficult to place the screws through the holes in the plate and into the proper place in the selected vertebrae. What is needed is an improved Casper pin that simplifies and improves the spinal surgical procedures.

### SUMMARY OF THE INVENTION

The inventive Casper pins and Casper pin tools that can be used during cervical spine, neck and back surgeries. The Casper pins are a single length and the insertion depths of the pins are controlled by the insertion tools. In an embodiment a number of different insertion wrenches are available to the surgeon, each of the insertion wrenches providing a different insertion depth of the inventive pin. In contrast, the prior art uses different threaded length Casper pins and a single tool to insert and remove the pins.

The single length Casper pins are also different than the prior art Casper pins. Rather than having a wide diameter nut secured to the front portion of the pin, the front of the inventive Casper pin has a uniform outer diameter. The back ends of the inventive Casper pins have a driving surface that engages a corresponding driving surface in the insertion wrench that is mounted within a tubular section of the insertion wrench. The insertion wrench is designed to only insert the Casper pin a specific insertion depth which is typically 14 mm, 16 mm or 18 mm. The insertion depth for each insertion tool is the length of the Casper pin minus the distance between the front edge of the insertion tool and the front edge of the driving surface. The insertion depth of each wrench is clearly marked and the wrench shaft can be color coded, for example, green=14, orange=16, red=18 mm on an outer surface of the wrench so that the surgeon can easily determine the proper wrench during the operation.

During the neck surgery a needle is inserted into the disc and an X-ray and/or MRI are taken for localization. From the X-ray and/or the MRI measuring tool, the surgeon can estimate or determine the vertebral anteroposterior (a-p) depth to be operated upon. The pins should be inserted as deep as possible into the vertebrae to obtain maximum purchase in bone while leaving a safe distance from the posterior cortex of the vertebra so as to not penetrate the spinal cord. The deeper vertebra holes will provide greater surface area for the Casper pins to separate the vertebra and also provide more surface area for the screw threads to hold the plate in place after the surgery is completed. Based upon the measurements the surgeon may take from the MRI “Measuring Tool” that is part of the MRI program or simply and eyeball measurement taken from the X-ray, the proper Casper pin insertion depth is determined. The insertion wrench corresponding to the proper insertion depth is selected and the Casper pin is inserted into the selected insertion wrench.

The pins are now placed into the vertebrae with the knowledge that the pins will serve as screw sites for the plate. As such, the pins are placed close to the vertebral end plate where bone is strongest. The pins are preferentially placed in parallel. The parallelism of the pins will serve as guide to “index” the proper plate length. The tip of the inventive Casper pins can have a bone cutting edge and a threaded outer surface so the tip of the pin is a self taping mechanism. By pressing the Casper pin against the vertebra and rotating the insertion tool clockwise, the Casper pin bores into the vertebra creating a hole having an internally threaded surface. As the Casper pin approaches the designated insertion depth, the front end of the insertion tool will contact the vertebra surface and the driving surface of the Casper pin will start to pull away from the driver of the insertion tool. When the Casper pin reaches the proper insertion depth, the driving surface separates from the driver in the insertion tool to prevent the pin from rotating or going any deeper into the vertebra. This process is repeated on a second vertebra that is also adjacent to the damaged disc.

Once the Casper pins are in place, a distractor is coupled to the Casper pins to separate the vertebrae for the discal removal. During the surgery the damaged disc can be removed and a cage or a disc replacement can be placed between the adjacent vertebrae. Alternatively, a bone graft can be used if a bone fusion is being performed. In other embodiments, the surgeon can choose to place another structure between the vertebrae. With the chosen structure in place, the distractor is removed and the vertebrae are tightly approximated to the cage as a result of muscle tension. The vertebra on either side of the cage, disc replacement or bone graft if a fusion is being preformed, are secured with a plate having fastening holes spanning the cage, disc or bone graft. A properly sized plate is selected by the surgeon based upon the distance between the two parallel Casper pins, thus avoiding the conundrum of determining the proper plate length based upon the vertebrae hole spacing which can be difficult to see due to the blood in this area.

The plate having the correct length is simply dropped over the uniform outer diameter pins. The plate then slides over the lengths of the pins down to the vertebrae. If the pins are not perfectly parallel and there is some divergence or convergence of the pins, simple finger pressure allows the plate to drop over the inventive pin which is now both a measuring guide for pin length and an insertion device, assuring the plate will be the proper length, in the proper place, with excellent screw purchase as a result of optimal screw placement and screw length. Some surgeons may desire a second X-ray, to determine if the cage and plate construct has been properly positioned. This second X-ray can also be used to determine if the insertion depth of the inventive Caspar pin is optimal. If the insertion depths are too short as seen on the X-ray, the inventive Casper pins can be inserted deeper into the vertebrae. Since the length of the inventive pin is known, an insertion wrench can be chosen to insert the Casper pin deeper into the vertebrae to the proper depth.

When the optimum screw depth is known, the proper screw length used to hold the plate to the vertebrae can be determined. For example, if the proper vertebrae hole depth is 16 mm and the plate thickness is 2 mm, then the proper screw length is 18 mm. The equation is screw length−plate thickness=actual screw insertion depth into the vertebra. After the plate is positioned against the vertebrae, the pins are removed in sequence and replaced with fastening screws chosen by the above method. The Casper pins are removed with a removal wrench that is similar to the insertion wrenches but has a shorter tubular section so the driver in the removal wrench can engage the driving surface of the fully inserted Casper pin. In an embodiment, the removal tool only allows the Casper pins to rotate in a counter clockwise direction so that the pin can only be removed from the vertebra. This assumes that the Casper pin has right handed threads, so that the counter clockwise only rotation is the removal direction. Because the tips of the Casper pins are very close to the posterior cortex and spinal cord it is critical that the removal tools cannot be used to unintentionally further insert the pins.

The Casper pins are replaced with fastening screws. The lengths of the screws are determined by the Casper pin insertion depths minus the plate thickness as above described. The screws are simply screwed into the holes formed in the vertebrae by the Casper pins that are now pilot holes for the screws. The outer diameter of the threads of the screws can be wider than the outer diameter of the Casper pins. After the plate is secured to the vertebrae with the tightened screws, the incision that exposed the spine can be closed to complete the neck surgery.

The inventive apparatus and method have many advantages over the prior art. The inventive method is much more efficient because there is no wasted time. The improved Casper pins are used to determine the proper plate length and the pins remain in place when the plate is positioned against the vertebrae. The optimum screw length is also easily determined and it is much easier to find the proper insertion hole when the plate is in place against the vertebrae. There is also no need to drill, bore, or make any other holes in the vertebrae, minimizing bleeding, and maximizing visualization For these reasons, the inventive Casper pin apparatus and vertebrae surgery method are significant improvements over the prior art.

### BRIEF DESCRIPTION OF THE DRAWINGS

FIG. 1 illustrates a Casper pin;

FIG. 2 illustrates a wrench used with the Casper pin;

FIG. 3 illustrates a distractor;

FIG. 4 illustrates a modified Casper pin;

FIGS. 5-9 illustrate different driving surfaces at the end of the Casper pin;

FIG. 10 illustrates an insertion wrench used with the modified Casper pin;

FIG. 11 illustrates a Casper pin inserted into the insertion wrench;

FIG. 12 illustrates an insertion wench and a Casper pin partially screwed into the vertebra;

FIG. 13 illustrates an insertion wench and a Casper pin fully screwed into the vertebra;

FIG. 14 illustrates two Casper pins fully inserted into adjacent vertebra;

FIG. 15 illustrates a distractor placed over the Casper pins to separate the vertebra;

FIG. 16 illustrates an 18 mm insertion tool further inserting the Casper pin;

FIG. 17 illustrates a plate placed over the Casper pins and a removal tool;

FIGS. 18 and 19 illustrate the ratchet mechanism of the removal tool;

FIG. 20 illustrates a top view of the plate;

FIG. 21 illustrates a side view of the plate;

FIG. 22 illustrates a screw holding the plate to the vertebra; and

FIG. 23 illustrates two screws holding the plate to the adjacent vertebra.

### DETAILED DESCRIPTION

The present invention is directed towards an improved Casper pin and special tools used to insert and remove the Casper pins from vertebrae. The tools include insertion wrenches that insert the Casper pins to specific insertion depths and a removal tool that can only be used to extract the Casper pins. The inventive insertion wrenches allows a single length pin to be inserted into a vertebra to any required insertion depth. The removal wrench only allows the Casper pin to be removed from the vertebra and may not be used to insert the Casper pins into the vertebra.

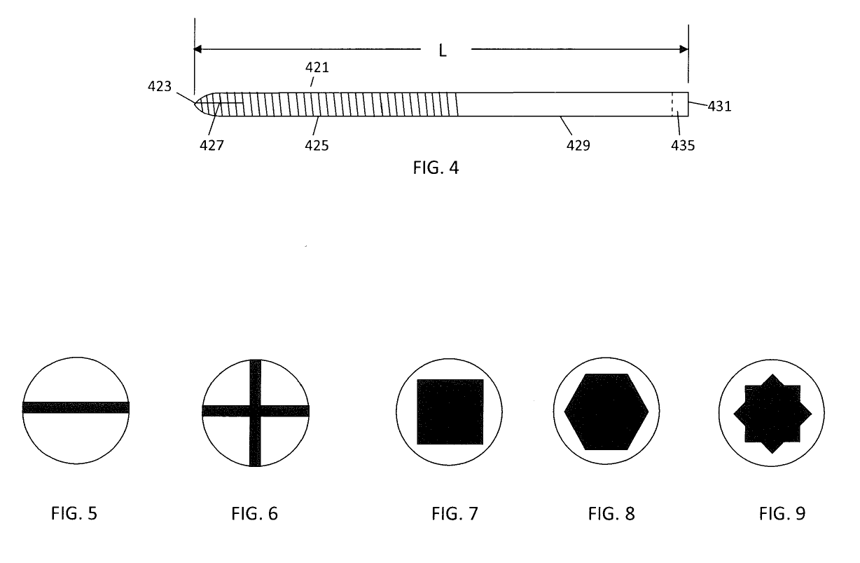

With reference to FIG. 4 the inventive Casper pin 421 includes a front tip 423, a substantially cylindrical body and a back end 431. The tip 423 may have a cutting surface 427 to assist in forming the holes and internal threads in the vertebrae. The pin 421 also has a threaded 425 outer diameter that extends from the tip 423 to the back portion 429. For example, the threads can extend from the tip 423 along the body of the pin 421 by 18 mm or more so the pin 421 can be screwed into the vertebra with an insertion depth anywhere between 12-18 mm. The outer diameter of the threaded section 421 may be larger than the outer diameter of the back portion 429. In another embodiment, the threads can extend along the entire outer diameter of the pin 421. The outer surface at the back portion 429 of the Casper pin 421 can be a smooth cylindrical surface or any other shape that does not increase the outer diameter dimension.

The Casper pins 421 can have a total length L that is between about 50 and 120 mm. In order to accurately control the insertion depth of the inventive Casper pins, the length of the pins 421 must be precisely controlled during manufacturing. In particular, the Casper pin cannot be any longer than the designated length since this would result in the actual insertion depth being deeper than expected. In order to prevent over insertion, the manufacturing tolerance for the inventive Casper pin length can be +0.00 mm and −0.02 mm.

A drive head 435 is located at the back end 431 of the pin 421 and has physical features that allow the pins 421 to be forcefully rotated and screwed into vertebrae. The drive head 435 can be any type of driving surface. For example, suitable drive heads include flat slot (FIG. 5), Philips (FIG. 6), square (FIG. 7), hex (FIG. 8), torx, double square (FIG. 9), spanner head, triple square, poly drive, double hex, Bristol, etc. The drive head features can be recessed surface similar to the surfaces of bolts or screws, or alternatively, the driving head 435 can be a protruding surface at the end of the pin 421 similar to the driving surface of a wench or screw driver. Because the Casper pins 421 are inserted a precise insertion depth into the vertebrae, the back edge of the drive heads are preferably flat and not tapered across the width of the pins 421. The corners at the intersection of the back surface 431 and the driving head 435 should also come to a sharp 90 degree edge with a minimum corner bevel or radius.

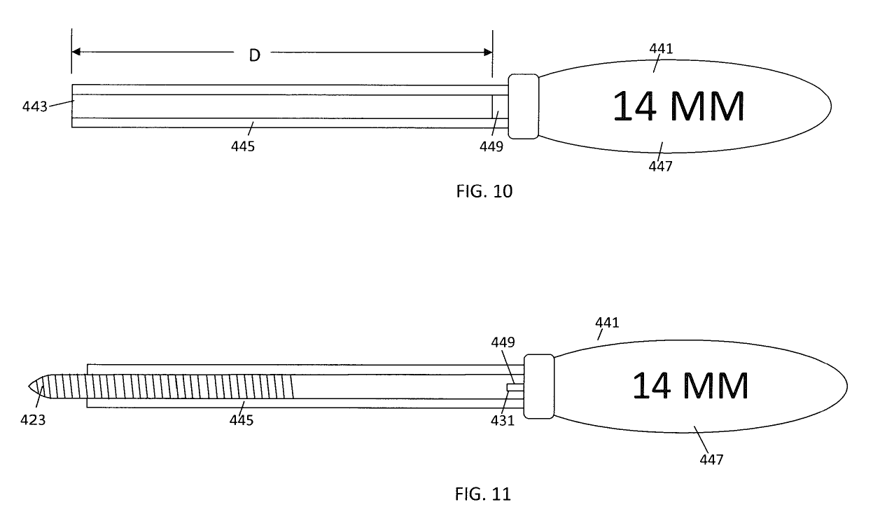

With reference to FIG. 10, a side view of an insertion wrench 441 used to install the inventive Casper pin is illustrated. The wrench 441 has a tubular portion 445 shown in cross section and a handle portion 447. The front end 443 of the tubular portion 445 is a smooth flat surface and the inner diameter of the tubular portion 445 is slightly larger than the outer diameter of the Casper pin. The tubular portion 445 holds the Casper pin in a proper alignment with the insertion wrench 441. The wrench 441 has a driver 449 within the tubular portion 445 that corresponds to the driving surfaces at the back end of the pin. In this example, the driving surface 449 is a flat blade shown in FIG. 5 that extends across the width of the inner diameter of the tubular portion 445. The driver 449 should have a straight and flat front surface that is perpendicular to the center axis of the wrench 441. The front edge of the driver 449 should not be tapered and the corners at the exposed edge should come to a sharp 90 degree edge with only a minimum edge bevel or radius. The depth D between the front end 443 of the wrench 441 and the front end of the driving surface 449 is critical to the proper insertion of the Casper pins and must be precisely controlled when the wrench 441 is manufactured. To prevent over insertion, the manufacturing tolerance of the depth D can be +0.05 mm and −0.00 mm.

In a preferred embodiment, the Casper pins 421 are used with one of several different insertion wrenches 441 each having a different insertion depth. The insertion depth is critical to the operation and the surgeon cannot make an error in the insertion depth. In order minimize the possibility of errors, the insertion depths are clearly marked on the insertion tools. The insertion depth marking can be a numerical insertion depth indicator. For example, the wrench illustrated in FIGS. 10-13 is marked “14 MM” on the handle portion and is used to insert the Casper pin to an insertion depth of 14 mm. The insertion depth text can be marked on multiple locations around the handle 447, so that the insertion depth is clearly visible no matter how the wrench 441 is placed on an instrument tray surface. The insertion depth of the insertion wrenches 441 can also be a color coding. For example: blue for 12 mm, green for 14 mm, orange for 16 mm, red for 18 mm, etc. While the normal insertion depths may vary by 2 mm in other embodiments, other insertion depth wrenches can be used such as 13 mm, 15 mm, 17 mm or any other insertion depth.

Based upon the equation D=L−Insertion Depth, if the wrenches are used with L=85 mm long Casper pins, the D distances for the different insertion depths for the different insertion depths are the lengths shown in Table 1 below. For example, a 12 mm insertion depth tool will have a D dimension=73 mm and a 16 mm insertion depth tool will have a D dimension=69 mm. In other embodiments, any other Casper pin length L can be used and the D dimensions of the insertion wrenches can be changed accordingly so the Insertion Depths are properly controlled.

TABLE 1

| | Insertion Depth | | | | | | |
|---|---|---|---|---|---|---|---|
| | 12 mm | 13 mm | 14 mm | 15 mm | 16 mm | 17 mm | 18 mm |
| D dimension | 73 mm | 72 mm | 71 mm | 70 mm | 69 mm | 68 mm | 67 mm |

While the inventive Casper pins are a direct replacement for the prior art Casper pins, the procedures required to use the inventive Casper pins are substantially different. A needle is inserted into the vertebra and the patient's spine is X-rayed and the vertebral anteroposterior (a-p) depth is known from an MRI measuring tool that is part of the MRI program. Alternatively, this needle insertion depth can be estimated based upon a viewing of the X-ray and or MRI. Based upon this information, the surgeon can determine the proper vertebral level to be operated upon and determine the insertion depth of the Casper pin. For example, the surgeon may determine that the patient requires the Casper pins to have an insertion depth of 14 mm.

Rather than selecting a Casper pin having the desired insertion depth for the patient, the surgeon selects uses any of the uniform length and outer diameter Casper pins 421 with an insertion wrench having an insertion depth that corresponds to the proper insertion depth. With reference to FIG. 11, a uniform pin 421 is inserted into a 14 mm wrench. In this example, a slot type driving surface is used and the driving surface 431 of the pin 421 engages the driving surface 449 at the back end of the cylinder 445. The surgeon may rotate the pin 421 so that the driver 449 properly seats into the driving surface 431 of the pin 421. In this example, the distance D between the end of the wrench 443 and the front edge of the driving feature 449 is 71 mm. The tip 401 of the pin 421 extends less than 14 mm from the front surface 443 of the wrench 441. A side view of the straight blade driving surface 449 that engages the slot driving surface 431 of the Casper pin 421. As discussed above, in other embodiments, various different pin and insertion wrench dimensions can be used.

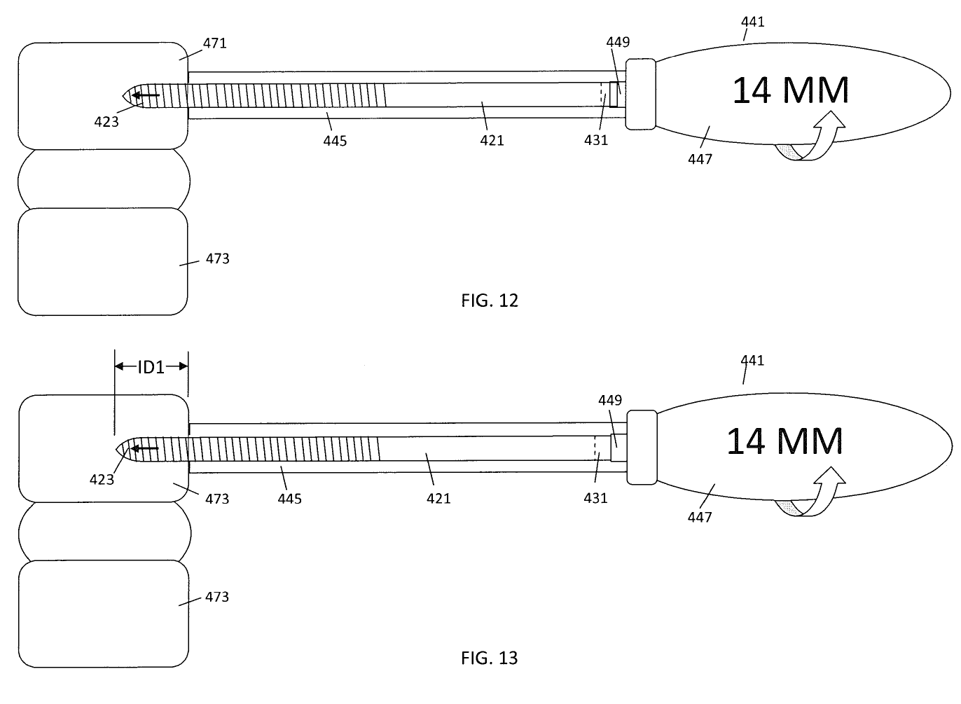

Before the Casper pins are inserted into the vertebrae, the surgeon may determine the positions of the Casper pins and the distance between the pins. Based upon this distance, the surgeon can determine the proper plate length that will be coupled to the vertebrae surrounding the disc that is being operated on. With reference to FIG. 12, the tip of the pin 423 is positioned over the insertion point of the vertebra 471 and the 14 mm wrench handle 447 is rotated to drive the pin 421 into the vertebra. As the pin is screwed into the vertebra 471, the front end 443 of the wrench 441 contacts the vertebra 471. The surgeon will continue to rotate the wrench 441 and the pin 421 will continue to screw into the vertebra 471. Since the wrench 441 is in contact with the vertebra 471, the driving surface 431 at the back end of the pin 421 will pull away from the driver 449 of the wrench 441.

With reference to FIG. 13, when the Casper pin 421 reaches the insertion depth “ID1,” the driving surface 431 of the pin 421 will completely pull away from the driving surface 449 of the wrench 441. In this example, the Casper pin 421 will be inserted to an insertion depth ID1 of exactly 14 mm. Further rotation of the wrench 441 will not cause the Casper pin 421 to rotate which prevents the Casper pin 421 from being screwed into the vertebra 471 any more than the set insertion depth of the wrench 441. After the Casper pin 421 separates from the insertion wrench 441, the 14 mm wrench 441 is removed from Casper pin 421.

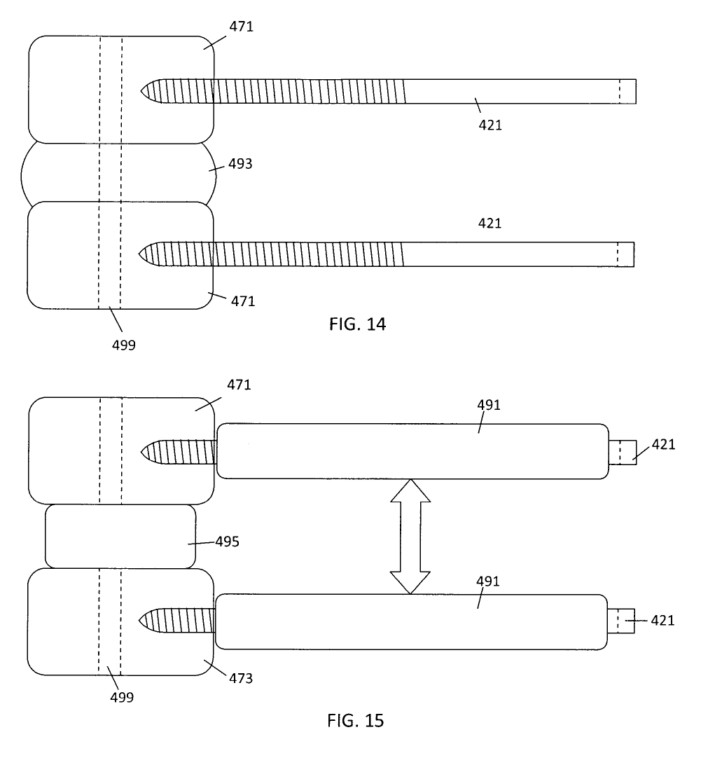

With reference to FIG. 14, the described process is repeated to insert a Casper pin 421 into the adjacent vertebra 473. The two Casper pins 421 are inserted to an insertion depth of exactly 14 mm into adjacent vertebrae 471, 473 on opposite sides of the damaged disc 493 and parallel. With reference to FIG. 15, a spreader tool is used to separate the vertebra 471. The tubes 491 of the spreader tool are placed over the pins 421 and the spreader mechanism separates the pins 421 and the adjacent vertebrae 471, 473. With the vertebrae 471, 473 separated, the surgeon can perform the discal removal. The Surgeon can replace the removed disc with a cage 495, bone graft, a disc replacement or any other chosen structure that is placed around the spinal cord 499 between the vertebrae 471.

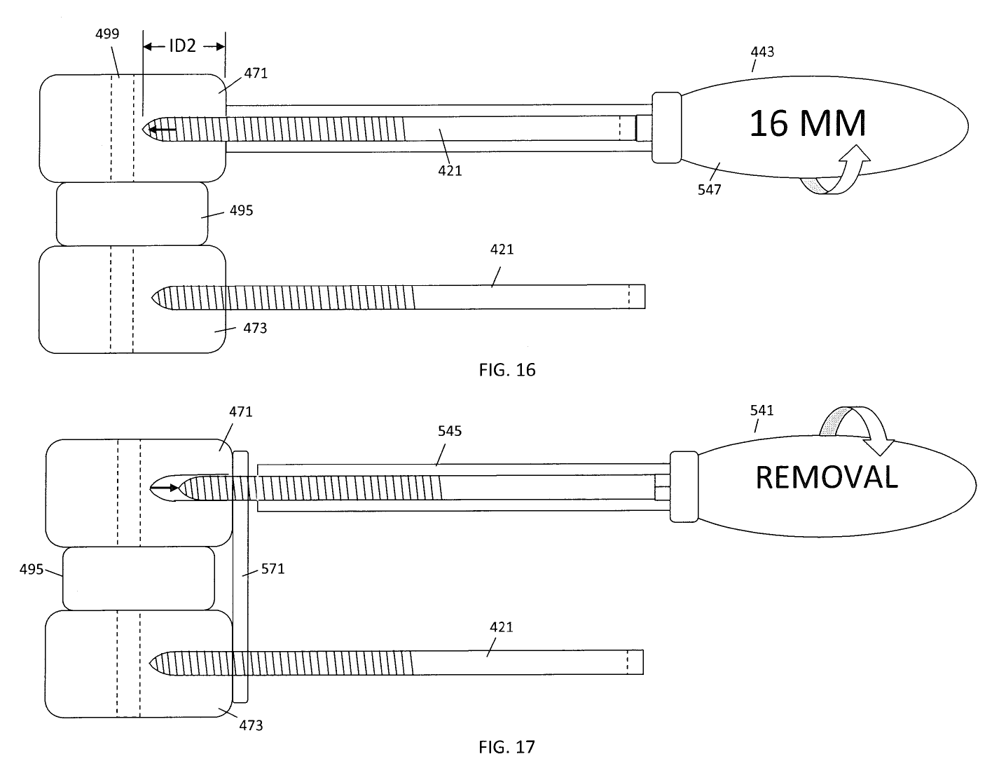

At this point, a second X-ray can be taken to determine if the cage 495 placement is correct. The X-ray will also show the position of the Casper pins 421 within the vertebrae 471, 473. The second X-ray can also provide additional information and may also indicate that the Casper pins 421 can be inserted deeper into the vertebra 471. For example, the x-ray may show that there is a distance of 4 mm between the front tip of the Casper pin 421 and the posterior cortex and spinal cord 499. With reference to FIG. 16, the surgeon will remember that a 14 mm insertion depth wrench was used to insert the Casper pin 421 to the first insertion depth ID1 of 14 mm. The surgeon will know that the posterior cortex and spinal cord 499 will not be contacted if the Casper pin 421 is moved 2 mm deeper into the vertebra 471. The surgeon can then select a 16 mm insertion depth wrench 442 and drive the Casper pins 421 an additional 2 mm deeper into the vertebra 471 to a 16 mm insertion depth, ID2. The ability to adjust the penetration depth of the inventive Casper pin 421 without removing the original Casper pin is a substantial benefit over the prior art Casper pins which each have a set non-adjustable insertion depth.

By knowing that the Casper pin 421 insertion depth is 16 mm, the surgeon can determine an appropriate screw length by the equation, screw length=screw purchase depth into vertebra+plate thickness. In this example, a 16 mm screw purchase depth into vertebra 471, 473+a 2 mm plate 571 thickness would result in a 18 mm screw length.

With the parallel pins 421 extending from the vertebrae 471, 473 the surgeon can select the plate 571 that corresponds to the distance between the parallel Casper pines 421. Alternatively, the surgeon can determine the proper plate length by measuring the distance between the pins 421 at the vertebrae 471,473. With the proper length plate selected, the plate slides over the lengths of the pins down to the vertebrae. With reference to FIG. 17, the cage 495 is in place and the plate 571 is placed over the Casper pins 421 against the vertebrae 471, 473. With the plate 571 properly positioned, the Casper pin 421 can be removed with a removal wrench 541. The removal wrench 541 has a tubular portion 545, a driving surface 549, and a handle 547 that are similar to the insertion wrench. However, the tubular portion 545 is slightly shorter that the insertion wrenches so that a single removal wrench 541 can be used for Casper pins 421 inserted to a depth of 12 to 18 mm by any insertion wrench. In a preferred embodiment, the removal wrench 541 must not be able to screw the Casper pins 421 deeper into the vertebrae 471, 473.

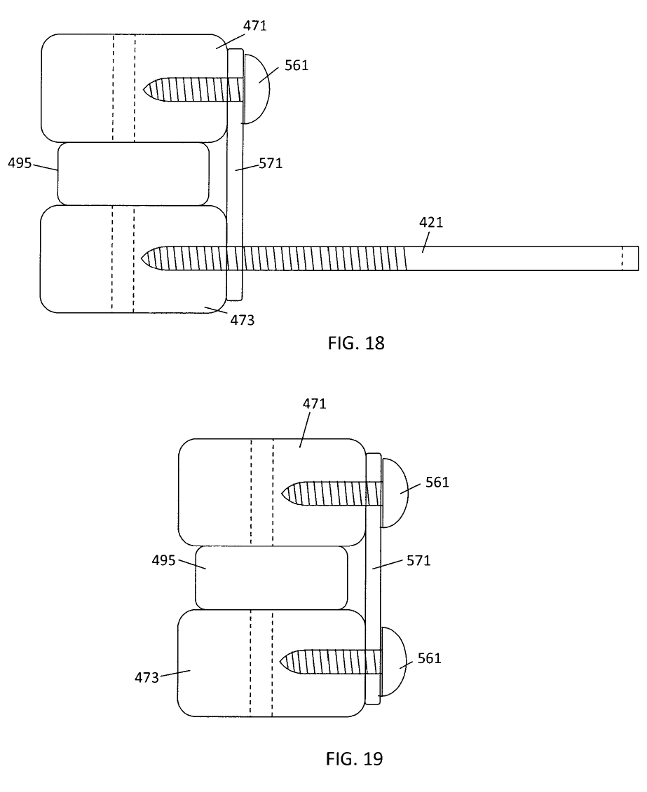

With reference to FIG. 18, the upper Casper pin 421 has been removed and replaced with a screw 561. The head of the screw 561 engages the top of the plate 571 so that the teeth at the bottom of the plate 571 are pressed into the vertebra 471. In this example, an 18 mm screw 561 is screwed into the vertebra 471 and used to securely hold the plate 571 is place. As discussed above, the Casper pin 421 was inserted to an insertion depth of 16 mm and the plate is 2 mm thick. The threads of the screw 561 can have a larger outer diameter than the threads of the Casper pin 421 to improve the engagement of the screw 561 with the vertebra 471. Thus, the holes formed in the vertebrae 471, 473 function as pilot holes for the threaded portion of the screw 561.

With reference to FIG. 19, the second Casper pin 421 is removed and replaced with a second 18 mm screw 561. The screws 561 are tightened to permanently secure the plate 571 to the adjacent vertebra 471, 473. The outer surfaces of the screws 561 and plate 571 are smooth and do not have any sharp edges. In the preferred embodiment, the Casper pin 421, plate 571 and screw 561 are made of inert materials such as stainless steel, titanium, ceramics, and composites, etc. When the surgery is complete, the incision cut into the patient to expose the spine can be closed so the spine is allowed to heal.

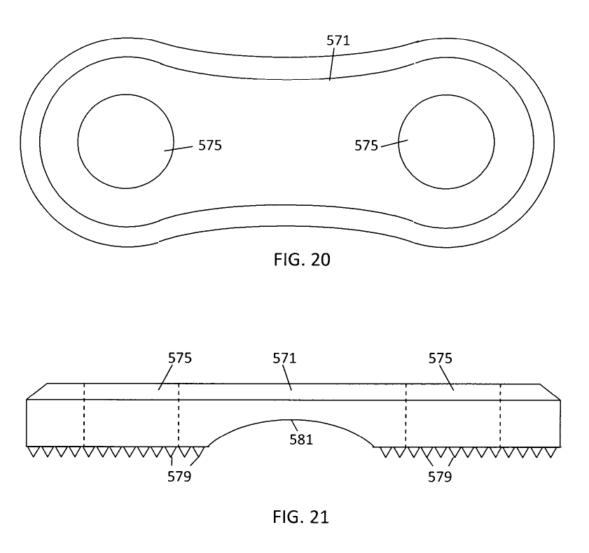

Various types of plates 571 can be used with the inventive Casper pins 421. FIG. 20 illustrates a top view and FIG. 21 illustrates a side view of an embodiment of a plate 571 that can be secured to the vertebrae. Two through holes 575 extend through the thickness of the plate 571. The upper outer edges of the plate 571 are beveled to remove any sharp edges. The bottom surface has a plurality of small teeth 579. When the plate 571 is secured to the vertebra, the teeth 579 are pressed into the vertebra to prevent the plate 571 from moving or rotating. The bottom surface of the plate 571 also has a recessed section 581 that is positioned across the gap over the cage 495 between the adjacent vertebrae. The plates 571 can be made in various different lengths between the two through holes 575. The surgeon can form the holes in the vertebrae 471, 473 based upon the plate 571 being used. Alternatively, the surgeon can measure the distance between the Casper pins 421 in the vertebrae 471, 473 to determine the proper sized plate 571 for the patient. Example of other suitable plates include: a uniplate or other mid-axial type plate can be placed over the Casper pins 421 and secured to the vertebrae 471, 473 with screws 561.

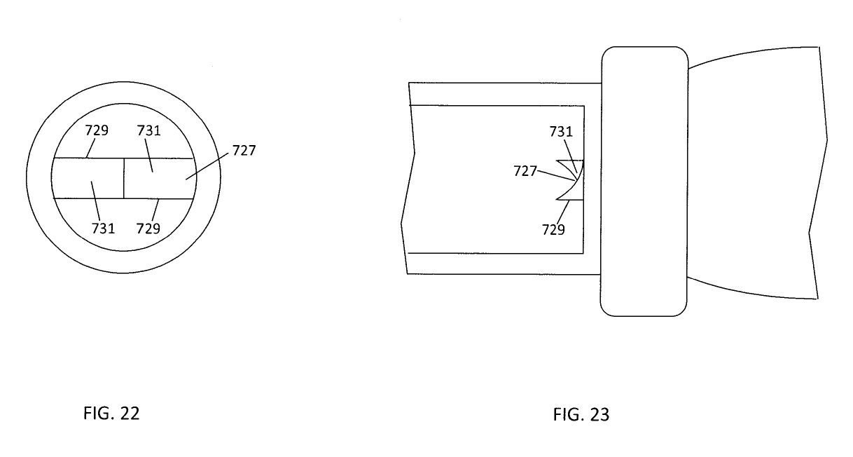

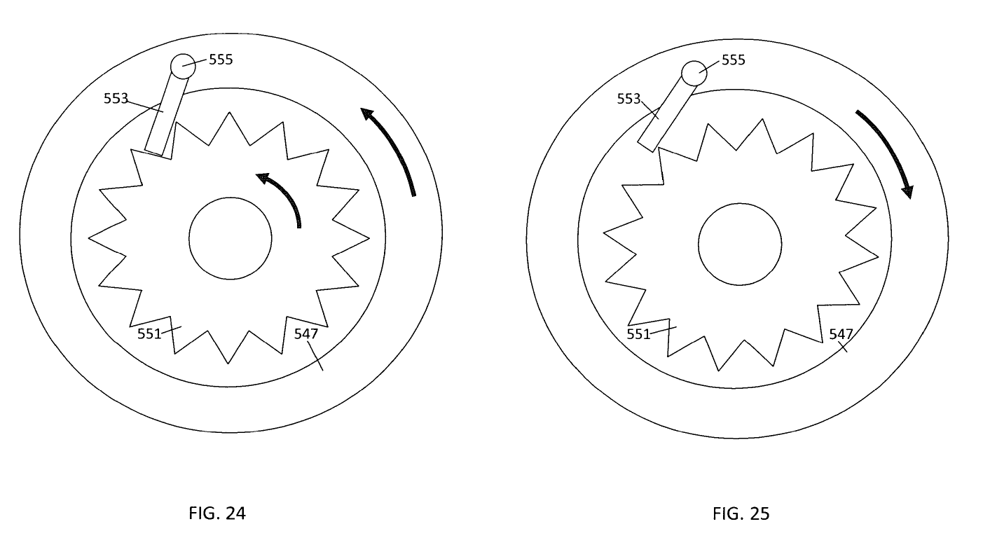

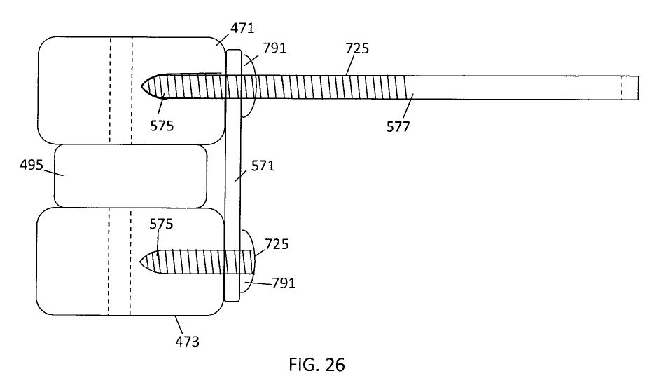

With reference back to FIG. 17, additional operating details of the removal wrench are disclosed. If the surgeon accidentally rotates the removal wrench handle 547 clockwise, the removal only drive mechanism 549 prevents any clockwise rotation of the Casper pin 421. This safety feature prevents the surgeon from accidentally driving the Casper pin 421 deeper into the vertebrae 471 which can damage the spinal cord 499. With reference to FIGS. 22 and 23 detailed views of an embodiment of the removal driver 727 are illustrated. FIG. 22 illustrates a front view and FIG. 23 illustrates a side view of the removal tool 541. The driver 727 is split into two sides. A sharp side 729 engages the slotted portion at the end of the Casper pin when the removal tool 541 is rotated counter clockwise. In contrast, when the removal tool 541 is rotated, clockwise, the ramped surface 731 will not engage the slot and the removal tool 541 will rotate without rotating the Casper pin.

In other embodiments, different mechanisms can be used prevent the removal wrench from being used to screw the Casper pins deeper into the vertebra. For example with reference to FIGS. 24 and 25, the removal wrench may include a ratchet mechanism that prevents the driver from rotating clockwise. In order to prevent further insertion, the removal wrench can have a ratchet mechanism that only rotates the Casper pin in a counter clockwise which is the removal rotation direction. The ratchet mechanism can have a pawl 553 and a gearwheel 551 coupled to the tubular portion 545. When the handle 547 is rotated counter clockwise, the pawl 553 engages the gearwheel 551 causing the driver and Casper pin to rotate. In contrast, when the handle 547 is rotated clockwise, the pawl 553 does not engage the gearwheel 551 and the driver and Casper pin do not rotate. This feature prevents the Casper pin from being screwed deeper into the vertebra. The removal tool driver can only be used to remove the Casper pin from the vertebra.

In a preferred embodiment, the insertion tools and removal tools each perform one function and are all clearly marked so the surgeon will know what each of the tools is used for. However, it is also possible to have a single tool that performs multiple tasks. For example, the adjustable wrench tool may have a mechanism that allows the insertion depth of the Casper pin to be adjusted. In this embodiment, the surgeon would identify the proper insertion depth and then adjust the tool to match the proper insertion depth. For example, the handle can have a screw mechanism that is used to change the distance between the front of the tubular section and the front of the driving surface can be set to the proper insertion depth of about 14-18 mm. The adjustment mechanism may also have a visual indicator that indicates the insertion depth setting and a locking mechanism that prevents the insertion depth from being changed. Thus, several insertion wrenches can be replaced by a single insertion tool.

In an embodiment, the adjustable Casper pin insertion tool may have an adjustable ratchet mechanism that allows the tool to be switched between the “insertion” mode described above and a “removal” mode that will only allow the Casper pin to rotate in a counter clockwise direction. In the removal mode, the distance between the front of the tubular section and the front of the driving surface is shortened, so the driving surface can engage the Casper pin regardless of insertion depth. A ratchet mechanism is also actuated to prevent the rotation of the driving mechanism in the clockwise direction as described above, and prevent the Casper pin from being driven deeper into the vertebra.

In the described procedure above, the Casper pins are used to create pilot holes and are used to position the plate over the vertebrae. The Casper pins are then removed and screws are screwed into the holes formed by the Casper pins. In other embodiments, a different Casper pin and procedure can be used to secure the plate to the vertebrae. For example, a multiple piece Casper pin can be used to secure the plate to the vertebrae. With reference to FIG. 26, two multiple piece Casper pins 725 are screwed into adjacent vertebrae as described above. The plate 571 is placed over the Casper pins 791 and moved against the vertebrae 471, 473. Rather than replacing the Casper pins with screws as described above with reference to FIGS. 18 and 19, nuts 791 can be screwed down on the threads of the Casper pins 725 to secure the plate 571 against the vertebrae 471, 473. Once the nuts 791 are in place against the plate 571, the multi-piece Casper pins 725 are separated. The inserted portion 575 remains in the vertebrae 471, 473 while the extending portion 577 is removed. Various known pin coupling mechanisms can be used to keep the multi-piece Casper pin 791 together before the extending portion 577 is separated from the inserted portion 575. When the surgery is complete, the incision cut into the patient can be closed so the spine is allowed to heal. The described steps replace the steps described and illustrated above with reference to FIGS. 17-19.

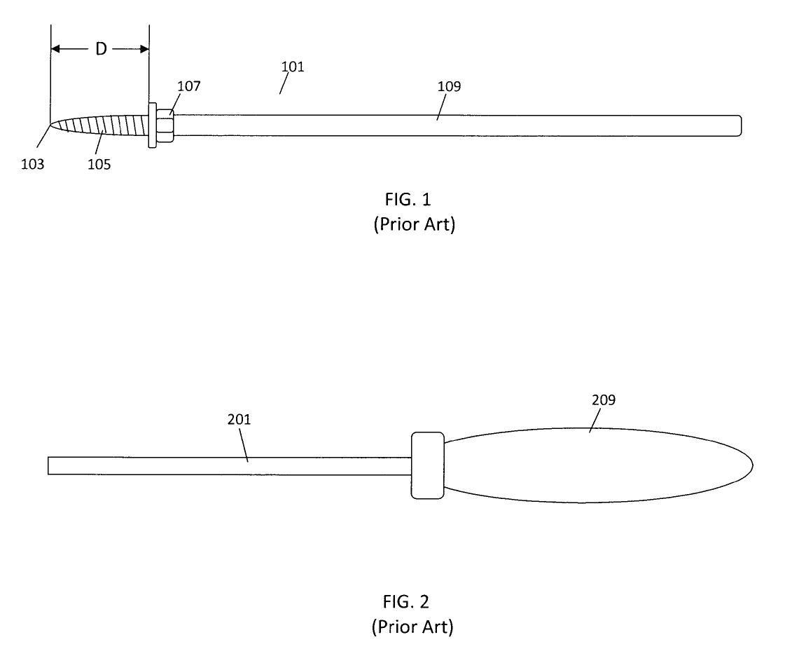

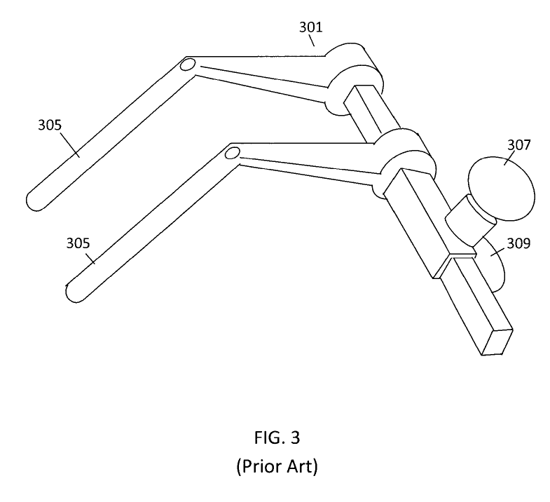

The inventive Casper pin and tools have various advantages over the prior art. A significant problem with the prior art, is that the Casper pins need to be removed before the plate can be attached with screws to the vertebra. With reference to FIG. 1, the holes in the plate cannot be placed over the prior art Casper pins 101 because of the hex nut 107 that is secured to the pins has a much wider diameter than the holes in the plate. Thus, the prior art Casper pins 101 must be removed before the plate can be secured with screws to the vertebra. It is very difficult to properly position the plate over the vertebra after the prior art Casper pins 101 have been removed, because of the bleeding in the surgery area. In contrast, the inventive Casper pin system greatly simplifies the process because the plate can be secured in place against the vertebrae with the Casper pins in place and does not need to be removed prior to permanently securing the plate to the vertebrae with screws. Thus, there is always a structural component for accurately positioning the plate over the vertebrae.

The prior art also do not allow the surgeon to make any Casper pin insertion depth adjustments during the surgery. If an insertion depth error is made, the surgeon must get a different length Casper pin and perform the insertion procedure again. In contrast, the insertion depth of the modified Casper pins can be adjusted by selecting a deeper insertion depth wrench without removing the pin.

It will be understood that the inventive system has been described with reference to particular embodiments, however additions, deletions and changes could be made to these embodiments without departing from the scope of the inventive system. For example, the same processes described can also be applied to other devices. Although the systems that have been described include various components, it is well understood that these components and the described configuration can be modified and rearranged in various other configurations.

What is claimed is:

1. A medical instrument set comprising:
at least two Casper pins adapted for insertion into vertebrae each Casper pin having a tapered point at a front end, a driving head at a back end, a threaded outer surface at the tapered point and a uniform length; and
at least two insertion tools each of the insertion tools having a tubular section, a driver within the tubular section for engaging the driving head of the Casper pin, and a handle for manually rotating the driver, each of the insertion tools having a different insertion depth defined by the length of the Casper pin minus a distance between front end of the insertion tool and a front surface of the driver.

2. The medical instrument set of claim 1 wherein the insertion depths of the insertion tools are between 12 mm and 18 mm.

3. The medical instrument set of claim 1 further comprising:
a removal tool having a tubular section, a removal driver and a handle for rotating the removal tool;
wherein a distance between a front surface of the removal tool and a front surface of the driver in the removal tool depth is less than the distances between front ends of the insertion tools and front surfaces of the drivers in the insertion tools.

4. The medical instrument set of claim 3 wherein the length of the Casper pin minus a distance between a front end of the removal tool and a front surface of the driver in the removal tool is less than 12 mm.

5. The medical instrument set of claim 3 wherein the removal tool includes a ratchet mechanism that only allows the driver to be rotated in one rotational direction.

6. The medical instrument set of claim 1 wherein the driving head of the Casper pin includes a slotted.

7. The medical instrument set of claim 1 wherein the driving head of the Casper pin includes a recessed area.

8. A method for performing a medical procedure on a patient comprising:
providing Casper pins each having a tapered point at a front end, a driving head at a back end, a threaded outer surface at the tapered point and a uniform length,
providing an insertion tool having a tubular section and a driver within the tubular section and a handle for rotating the insertion tool;
inserting a first Casper pin into the cylindrical body of the insertion tool so the driver engages the driving head;
driving the first Casper pin into the vertebra by rotating the insertion tool;
contacting the vertebra with a front surface of the insertion tool;
releasing the driving head of the first Casper pin from the driver of the insertion tool when the threaded point reaches a first insertion depth; and
removing the insertion tool from the first Casper pin.

9. The method of claim 8 further comprising:
placing a hole in a plate around the first Casper pin; and
moving the plate into contact with the vertebra.

10. The method of claim 8 further comprising:
removing the first Casper pin from a hole formed in the vertebra with a removal tool;
inserting a screw through the hole in the plate; and
screwing the screw into a hole formed in the vertebra to secure the plate to the vertebra.

11. The method of claim 8 further comprising:
providing a second insertion tool having a tubular section and a driver within the tubular section and a handle for rotating the second insertion tool;
placing the second insertion tool over the first Casper pin;
releasing the driving head of the first Casper pin from the driver of the insertion tool when the threaded point reaches a first insertion depth;
driving the first Casper pin into the vertebra by rotating the second insertion tool;
contacting the vertebra with a front surface of the tubular section of the second insertion tool;
inserting the first Casper pin into the vertebra to a second insertion depth using the second insertion tool.

12. The method of claim 8 further comprising:
inserting a second Casper pin into the cylindrical body of the insertion tool so the driver engages the driving head;
driving the second Casper pin into a second vertebra by rotating the insertion tool;
contacting the second vertebra with the front surface of the insertion tool;
releasing the driving head of the second Casper pin from the driver of the insertion tool when the threaded point reaches the first insertion depth; and
removing the insertion tool from the second Casper pin.

13. The method of claim 12 further comprising:
placing a first hole in a plate around the first Casper pin;
placing a second hole in the plate around the second Casper pin; and
moving the plate into contact with the second vertebra.

14. The method of claim 13 further comprising:
removing the second Casper pin from a hole formed in the second vertebra with a removal tool;
inserting a screw through the second hole in the plate; and
screwing the screw into a hole formed in the second vertebra to secure the plate to the second vertebra.

15. An insertion tool for inserting a Casper pin into a vertebra comprising:
a tubular section having a circular cross section;
a driver mounted within the tubular section for engaging a driving head of a Casper pin defining an insertion depth that is a uniform length of the Casper pin minus a distance between a front end of the insertion tool and a front surface of the driver; and
a handle for manually rotating the insertion tool.

16. The insertion tool of claim 15 wherein the driver of the insertion tool is released from the driving head of the Casper pin after a front surface of the insertion tool contacts the vertebra.

17. The insertion tool of claim 15 wherein the driver of the insertion tool is released from the driving head of the Casper pin after the Casper pin reaches an insertion depth into the vertebra.

18. The insertion tool of claim 15 wherein the insertion tool cannot insert the Casper pin deeper than the insertion depth.

19. The insertion tool of claim 17 wherein the insertion depth is between about 12 and 16 mm.

20. The insertion tool of claim 15 wherein the insertion tool cannot be used to remove the Casper pin from the vertebra.
